(19) United States
(12) Patent Application Publication
(10) Pub. No.: US 2023/0301875 A1
(43) Pub. Date: Sep. 28, 2023

(54) SYSTEM AND METHOD FOR PREPARATION AND DELIVERY OF RE-GENERATIVE FIBRIN MATRICES

(71) Applicants: Nickalis J. Dumas, Rossford, OH (US); Don M. Smucker, Coldwater, MI (US)

(72) Inventors: Nickalis J. Dumas, Rossford, OH (US); Don M. Smucker, Coldwater, MI (US)

(21) Appl. No.: 18/127,209

(22) Filed: Mar. 28, 2023

Related U.S. Application Data
(60) Provisional application No. 63/324,223, filed on Mar. 28, 2022.

(51) Int. Cl. A61J 1/20

(52) U.S. Cl. CPC A61J 1/2093

(57) ABSTRACT

The present invention of the system discloses a clear glass or plastic cylinder filled with small frosted glass beads. The cylinder is connected to two syringes by luer locks. The syringes force the blood sample through the cylinder running the sample over the frosted beads. The process includes rupturing of blood platelets based on contact between the surface area of the frosted glass rod and the blood, reducing processing time is as simple as increasing surface contact and increasing shear mixing.

### FIELD OF THE INVENTION

The present invention relates to a system or a method for creation of fibrin matrices. More particularly, the present invention discloses a system or a method to develop a quicker alternative for creation of the fibrin matrices.

### BACKGROUND OF THE INVENTION

Generally, fibrinogen is a major plasma protein, which participates in the blood coagulation process. Upon blood vessel injury, fibrinogen is converted to insoluble fibrin which serves as the scaffold for a clot. Blood coagulation of is a complex process comprising the sequential interaction of a number of plasma proteins, in particular of fibrinogen (factor 1), prothrombin (factor II), factor V and factors VII-XIII. Other plasma proteins such as Von Willebrand factor, immunoglobulins, coagulation factors and complement components also play a part in the formation of blood clots.

Fibrin is known in the art as a tissue adhesive medical device useful for wound healing and tissue repair. Lyophilized plasma-derived protein concentrate comprising fibrinogen, Factor XIII and fibronectin, in the presence of thrombin and calcium ions forms an injectable biological sealant such as passes through the cylinder fibrin glue.

The prior arts discuss on developing a quicker alternative for creation of fibrin matrices. The process discussed in the prior art involves using a hula cup to rupture platelet membranes releasing growth factors that create the fibrin matrix. This process currently takes ˜1 minute and anywhere from 20-60 ml of blood.

Since the process discussed in the prior art involves rupturing blood platelets based on the contact between the surface area of the frosted glass rod and the blood, reducing processing time is as simple as increasing surface contact to rupture platelets faster and/or increasing shear mixing that increases surface contact by better mixing.

Therefore, the improved process involves replacing the hula cup with a clear glass or plastic cylinder filled with small frosted glass beads. This cylinder will be connected to two syringes by luer locks. The syringes force the blood sample through the cylinder running the sample over the frosted beads. As the beads have a small diameter, the total surface area of contact can be increased in the cylinder by adding more beads. In addition to this, the process utilizes the plungers in the syringes to force the sample through the capsule or the cylinder, which is significantly more efficient way of mixing.

It is apparent now that numerous methods and systems are developed in the prior art that are adequate for various purposes. Furthermore, even though these inventions may be suitable for the specific purposes to which they address, accordingly, they would not be suitable for the purposes of the present invention as heretofore described. Thus, there is a need to provide a system or a method to develop a quicker alternative for creation of the fibrin matrices.

### SUMMARY OF THE INVENTION

In accordance to the present invention, the primary objective of the present invention is to develop a quicker alternative a system for creation of fibrin matrices. The system for creations and delivery of re-generative fibrin matrices is described here. The system involves rupturing blood platelets based on the contact between the surface area of the frosted glass rod or and the blood, reducing processing time is as simple as increasing surface contact to rupture platelets faster and/or increasing shear mixing that increases surface contact by better mixing.

Another objective of the present invention is to develop a system for preparation and delivery of Re-Generative Fibrin Matrix, the system includes a cylinder, a three-way stopcock and plurality of syringes. The cylinder includes a first end and a second end and filled with a plurality glass beads. Further, the cylinder includes a male luer lock on each of the first end and the second end.

The three-way stopcock includes a first way, a second way and a three way. Furthermore, the first way of the three-way stopcock is connected to the male luer lock on the first end of the cylinder.

Moreover, the plurality of syringes includes a first syringe, a second syringe and a third syringe. The first syringe is connected to the male luer lock on the second end of the cylinder. The second syringe is connected to the second way of the three-way stopcock and the third syringe is connected to the third way of the three-way stopcock.

Yet another objective of the present invention is to develop a method for preparation of Re-Generative Fibrin Matrix, the method comprises following steps:

A drawn blood syringe is connected to the cylinder. Then the stopcock is turned towards the third syringe. All of the air is drawn out of the cylinder with beads through second syringe pulling down blood through the cylinder till it reaches second syringe.

Then the stopcock is turned towards the second syringe allowing the third syringe to draw blood from cylinder and the first syringe. The last step is reversed sends the blood from the third syringe back into the cylinder and then into the first syringe. The process is repeated between the first syringe and the third syringe for 10 number passes through the cylinder. The platelet membranes in the blood are broken allows the release of fibrinogen to begin its formation to fibrin to form matrix blood.

Later, the stopcock is turned towards the cylinder. The matrix blood is flown into the second syringe with air which was drawn from the cylinder. The entire syringe is rested on a flat surface for 10 minutes once the matrix blood is passed into the second syringe completely.

The system is raised to allow air and matrix in the second syringe to separate air on the top and matrix at the bottom. The stopcock is opened to the cylinder and air is forced back into the cylinder till all the air is removed from the second syringe.

The stopcock is moved towards the cylinder allowing matrix to pass from the second syringe into the third syringe. Then, the cylinder and the first syringe are disconnected at luer lock connect leaving the second and third syringes connected to the stopcock.

The matrix is forced between the second syringe and the third syringe while matrix is mixed and rested with air. The fibrin chains breaks into shorter lengths to form the re-generative fibrin matrix and the Re-Generative Fibrin Matrix is delivered through the second syringe.

Other objectives and aspects of the invention will become apparent from the following detailed description, taken in conjunction with the accompanying drawings, which illustrate, by way for example, the features in accordance with embodiments of the invention.

To the accomplishment of the above and related objects, this invention may be embodied in the form illustrated in the accompanying drawings, attention being called to the fact, however, that the drawings are illustrative only, and that changes may be made in the specific construction illustrated and described within the scope of the appended claims.

Although, the invention is described above in terms of various exemplary embodiments and implementations, it should be understood that the various features, aspects, and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus, the breadth and scope of the present invention should not be limited by any of the above-described exemplary embodiments.

The presence of broadening words and phrases such as “one or more,” “at least,” “but not limited to” or other like phrases in some instances shall not be read to mean that the narrower case is intended or required in instances where such broadening phrases may be absent.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles. Furthermore, the drawings may contain text or captions that may explain certain embodiments of the present invention. This text is included for illustrative, non-limiting, explanatory purposes of certain embodiments detailed in the present invention. In the drawings:

Embodiments of the invention are described with reference to the following figures. The same numbers are used throughout the figures to reference like features and components. The features depicted in the figures are not necessarily shown to scale. Certain features of the embodiments may be shown exaggerated in scale or in somewhat schematic form, and some details of elements may not be shown in the interest of clarity and conciseness.

FIG. 1(A) illustrates a system for preparation of Re-Generative Fibrin Matrix in accordance with the present invention;

FIG. 1(B) illustrates a system architecture for preparation of Re-Generative Fibrin Matrix in accordance with the present invention;

FIG. 2(A) illustrates movement of a three-way stopcock attached to the cylinder in a first direction in accordance with the present invention;

FIG. 2(B) illustrates movement of the three-way stopcock attached to the cylinder in a second direction in accordance with the present invention;

FIG. 2(C) illustrates movement of the three-way stopcock attached to the cylinder in a third direction in accordance with the present invention; and

FIG. 3 illustrates a method for preparation of Re-Generative Fibrin Matrix in accordance to the present invention.

### DETAILED DESCRIPTION OF THE INVENTION

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In the description and claims of the application, each of the words “units” represents the dimension in any units such as centimeters, meters, inches, foots, millimeters, micrometer and the like and forms thereof, are not necessarily limited to members in a list with which the words may be associated.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

Regarding applicability of 35 U.S.C. § 112, ¶6, no claim element is intended to be read in accordance with this statutory provision unless the explicit phrase “means for” or “step for” is actually used in such claim element, whereupon this statutory provision is intended to apply in the interpretation of such claim element.

Furthermore, it is important to note that, as used herein, “a” and “an” each generally denotes “at least one,” but does not exclude a plurality unless the contextual use dictates otherwise. When used herein to join a list of items, “or” denotes “at least one of the items,” but does not exclude a plurality of items from the list. Finally, when used herein to join a list of items, “and” denotes “all of the items of the list.”

The following detailed description refers to the accompanying drawings. Wherever possible, the same reference numbers are used in the drawings and the following description to refer to the same or similar elements. While many embodiments of the disclosure may be described, modifications, adaptations, and other implementations are possible. For example, substitutions, additions, or modifications may be made to the elements illustrated in the drawings, and the methods described herein may be modified by substituting, reordering, or adding stages to the disclosed methods. Accordingly, the following detailed description does not limit the disclosure. Instead, the proper scope of the disclosure is defined by the appended claims. The present invention contains headers. It should be understood that these headers are used as references and are not to be construed as limiting upon the subjected matter disclosed under the header.

This specification includes references to “one embodiment” or “an embodiment.” The appearances of the phrases “in one embodiment” or “in an embodiment” do not necessarily refer to the same embodiment. Particular features, structures, or characteristics may be combined in any suitable manner consistent with this disclosure.

The presence of broadening words and phrases such as “one or more,” “at least,” “but not limited to” or other like phrases in some instances shall not be read to mean that the narrower case is intended or required in instances where such broadening phrases may be absent.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred, systems and methods are now described.

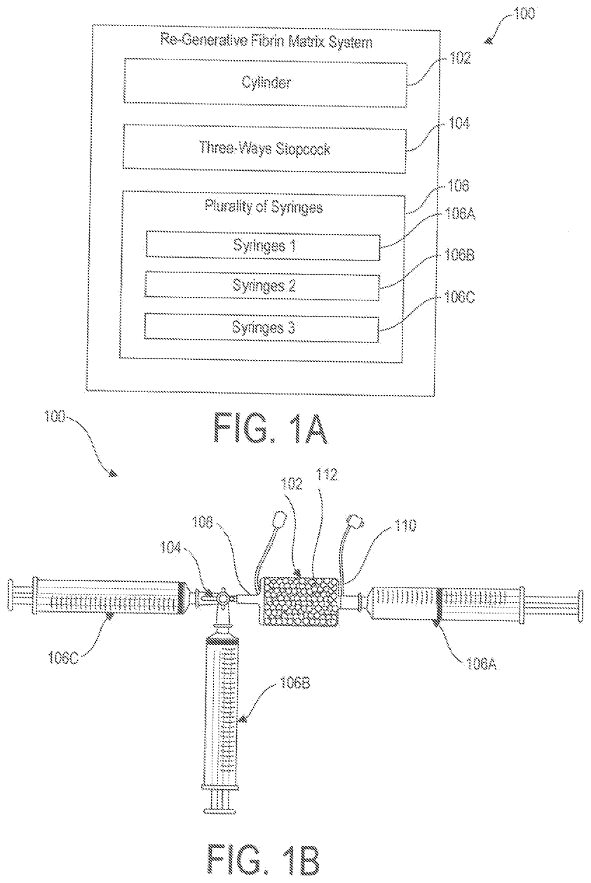

FIG. 1(A) illustrates a system diagram for preparation of Re-Generative Fibrin Matrix in accordance to the present invention. The system 100 for preparation and delivery of Re-Generative Fibrin Matrix is described/The system 100 includes a cylinder 102, a three-way stopcock 104 and plurality of syringes 106.

The cylinder 102 includes a first end 108 and a second end 110 and filled with plurality glass beads 112. Further, the cylinder 102 includes a male luer lock 114A, 114B on each of the first end 108 and the second end 110.

Further, the system 100 includes the three-way stopcock 104 includes a first way, a second way and a three way. Furthermore, the first way of the three-way stopcock 104 is connected to the male luer lock 114A on the first end 108 of the cylinder 102.

Moreover, the plurality of syringes 106 includes a first syringe 106A, a second syringe 106B and a third syringe 106C. The first syringe 106A is connected to the male luer lock 114B on the second end 110 of the cylinder 102. The second syringe 106B is connected to the second way of the three-way stopcock 104 and the third syringe 106C is connected to the third way of the three-way stopcock 104.

The cylinder 102 includes a clear glass or plastic cylinder filled with small frosted glass beads 112. This cylinder 102 connected to two syringes 106A, 106C by luer locks. The syringes will force the blood sample through the cylinder running the sample over the frosted beads. Because the beads have a small diameter, the total surface area of contact can be increased in the cylinder by adding more beads. To add to this the process of using the plungers in the syringes to force the sample through the capsule is a significantly more efficient way of mixing.

The current bead 112 size has been determined to be 0.25 in diameter meaning the single bead surface areas is 0.196 in and is defined by: A=4π r 2 where A is the surface area (in squared inches) of the frosted beads and r is the glass beads radius. With this known, it can now determine that the number of beads needed to match the surface area of the frosted glass rod is 29 beads.

The true time efficiency gained with this new improved process is by determining the surface area gained from the total number of beads that can be added to the glass cylinder. To determine the bead count we must start with interior volume of the glass cylinder.

The current glass cylinder is defined as being 2.5 inches in height and 0.5 inches in diameter. This means the interior volume is 0.491 in and is defined by: V=π r 2 h where V is the interior volume of the cylinder (in cubed inches), r is the cylinders measured radius, and h is the measured height of the cylinder. Next, we need to determine the volume of the glass beads. This volume was calculated to be 0.00818 in3 and is defined by: V=(4/3)π r 3 Where V is the volume of the sphere bead (in cubed inches) and r is the beads measured radius. With these two volumes known we can now determine the number of beads that can be placed in the capsule.

The total number is 136 beads and is defined by the following equation: N≤( )/((3 √2)) Where N is the Max number of beads that could fit in the cylinder, Vc is the volume of cylinder, and Vs is the volume of the sphere. Knowing that 136 beads fit in the cylinder we can determine that the surface area gained is 4.7× that of the hula cup. This means that 15 seconds is needed to match the same total surface contact gained during the 1 minute of mixing during the original process.

FIG. 1(B) illustrates a system 100 for preparation of Re-Generative Fibrin Matrix in accordance to the present invention. A cylinder 102 with beads in which the beads are sintered or have a roughened surface with a negative or positive charge covering the surface. A cylinder 102 having beads 112 within and having a male lug lock at each end 108, 110 of the barrel or the cylinder 102.

The cylinder 102 with sintered beads with which the two strings connected at either end of cylinder 102 with the male luer lock 114 at each end 108, 110 of the cylinder. The syringes 106A, 106B, 106C purpose is for transferring the blood through and over the beads so as to fracture the membrane of the platelets within the blood (First, second and third syringes).

A 3-way stopcock 104 is connected at one end 108 of cylinder 102 between male luer lock 114 and the female luer lock the syringe 106A, 106B, 106C. This can be opened to draw air into syringe attached to the 3 way stop cock.

The bead 112 size in diameter is specific to the count of the number beads to fracture the precise number of platelets when a specific number of passes are made with the blood product through the cylinder 102.

At the end of the each cycle of passing of the blood between the two syringes through the attached cylinder 102 with sintered beads 112 and the three-way stopcock 104, all blood products are transferred into the attached syringe without the stopcock 104. Then the stopcock 104 is opened to draw equal volume of air into the syringe attached to stopcock 104. Once the air has filled the empty syringe the stopcock is closed and then the air is forced through the stopcock 104 and cylinder 102 into the syringe 106B holding the blood products. When completed the whole system is placed on a flat surface for a specific period of time allowing the fibrinogen to form fibrin chains forming a modified clot.

After a specific amount of time has passed allowing for the formation to fibrin within the blood products. The cylinder 102 and stopcock 104 are removed from between these two syringes and is replaced with a double-end male luer lock with a specific portal size diameter of pathway which will break up long chain of fibrin into shorter smaller chains so as to be able to inject the liquefied fibrin matrix through smaller needle, i.e. 23-27-gauge needles.

When the fibrin matrix has been liquefied after a specific number of passes, the blood product can be mixed with other materials to aid in the healing process. However, this matrix must be injected within 30-60 seconds of liquefaction before the fibrin chains reconnect and become impossible to inject.

Benefits of the fibrin matrix is that it acts like a timed release of grow factors and other healing signaling which sustains signaling to other tissue cells for prolonged healing response. This autologous fibrin matrix accelerates the body's ability to heal itself by concentrating the healing signaling in a determined place generally with a needle. However, this fibrin matrix can also be solidified into a solid mass for a physical placement with a larger application apparatus for healing.

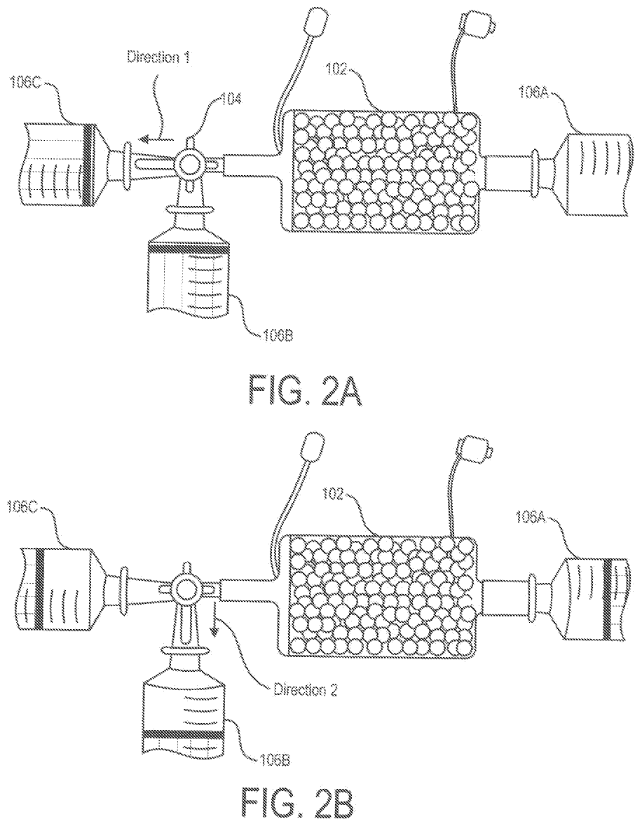

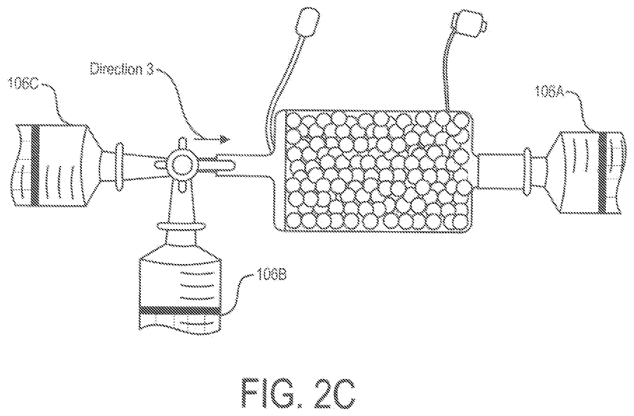

FIG. 2(A), FIG. 2(B) and FIG. 2(C) illustrates movement of a three-way stopcock 104 attached to the cylinder 102 in different directions in accordance to the present invention. The three-way stopcock 104 is attached two syringes (second syringe and third syringe) along with the cylinder 102. In FIG. 2(A) i.e. direction 1 the stopcock 104 is turned towards third syringe, used to draw all of the air out of the cylinder with beads, pulling down blood through the cylinder till it reaches second syringe.

At which point the three-way stopcock 104 is then turned towards second syringe i.e. direction 2 allowing third syringe to draw blood from cylinder 102 and first syringe 106A. When all of the blood is pulled out of first syringe into the cylinder 102 and into third syringe 106C the reverse process is reversed sending blood from third syringe 106C back into the cylinder 102 then into first syringe 102A. The reverse flow process is repeated between first syringe 106A and third syringe 106C for 10 number passes through the cylinder 102 housing the sintered beads 112. This process sheers or breaks the platelet membranes allow the release of fibrinogen to begin its formation to Fibrin.

Once the blood has passed 10 times between first 106A and third syringes 106C, then the stopcock 104 is turned towards the cylinder 102 i.e. direction 3 to flow the matrix blood into second syringe 106B with air which was drawn from housed within from the cylinder 102 at the beginning of this process. After the matrix blood is passed into the second syringe 106B completely, the entire syringe system 100 is allowed to rest on a flat surface for 10 minutes. This resting time allows the transformation of the fibrinogen to Fibrin, and thus creating a Fibrin matrix from the blood.

The Fibrin matrix is similar to a blood clot which is part of the healing component of blood in nature. After 10 minutes has passed and the matrix is solidified, then the system 100 is raised to allow air and matrix in the second syringe to separate air on the top and matrix at the bottom. The stopcock 104 is opened to cylinder 102 and air is forced back into the cylinder 102 till all the air is removed from the second syringe. Then, the stopcock 104 is opened allowing matrix to pass from second syringe into third syringe.

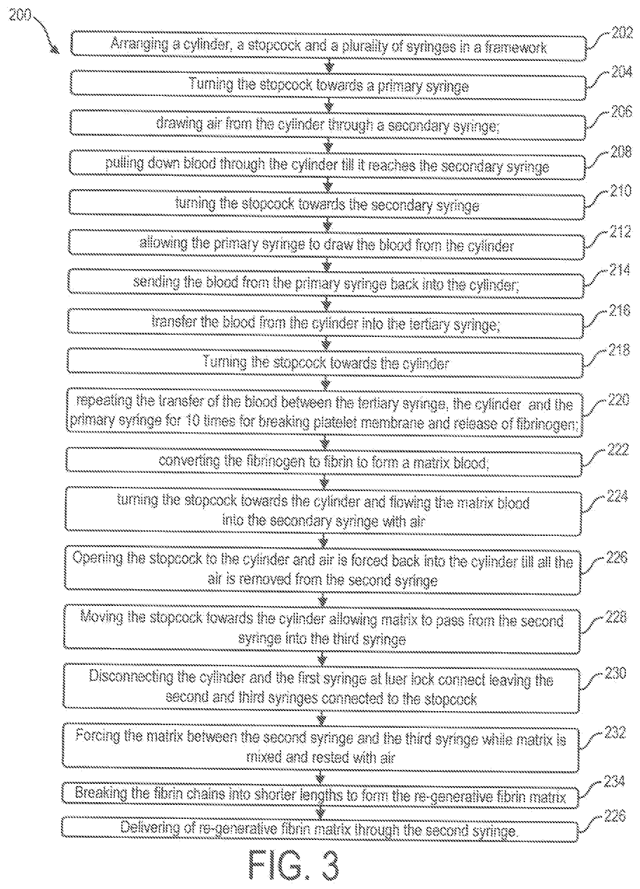

FIG. 3 illustrates a method 200 flowchart for preparation of Re-Generative Fibrin Matrix in accordance to the present invention. A sample of minimum of 20 ccs to 60 cc's or more of patients own blood is drawn into a large syringe. The syringe containing drawn blood or the blood sample is then connected to the cylinder 102 with sintered beads 112. At the other end of the cylinder 102, a three-way stopcock 104 is connected to the male luer lock. Attached to the remaining two luer lock ends of the three-way stopcock are attached two syringes or a primary syringe, a secondary syringe and a tertiary syringe of equal volume to that the syringe holding the patient's blood. For Example: if 20 cc syringe is used to draw 20 ccs of a patient's blood, there's 2-20 cc syringe would be attached to the three-way stopcock.

A method for preparation of Re-Generative Fibrin Matrix is described in FIG. 3. At step 202, a cylinder, a stopcock and a number of syringes in a framework is arranged. Further, at step 204, the stopcock is turned towards a primary syringe. Further, at step 206, air is drawn from the cylinder through a secondary syringe. Further, at step 208, the blood is pulled down blood through the cylinder till it reaches the secondary syringe. Further, at step 210, the stopcock is turned towards the secondary syringe. Further, at step 212, the primary syringe is allowed to draw the blood from the cylinder. Further, at step 214, the blood is sent from the primary syringe back to the cylinder. Further, at step 216, the blood is transferred from the cylinder into a tertiary syringe.

Further, at step 218, the transfer of the blood is repeated between the tertiary syringe, the cylinder and the primary syringe for ten times for breaking platelet membrane and for releasing fibrinogen. Further, at step 220, the fibrinogen is converted to fibrin for forming a matrix blood. Further, at step 222, the stopcock is turned towards the cylinder. Further, at step 224, the matrix blood is flowed into the secondary syringe with air. Further, at step 226, the framework is placed on a flat surface for ten minutes once the matrix blood is passes completely in the secondary syringe. Further, at step 228, the framework is uplifted to allow mixing of air and the matrix blood in the secondary syringe. Further, at step 230, the air is separated from the top end and the matrix blood at the bottom end of the secondary syringe.

Further, at step 232, the stopcock is opened to the cylinder for forcing air back into the cylinder till the air in the secondary syringe is removed. Further, at step 234, the stopcock is turned towards the cylinder to allow the matrix blood. Further, at step 236, the matrix blood is passed from the secondary syringe to the primary syringe. Further, at step 238, the cylinder and the tertiary syringe is disconnected at a luer lock connect. Further, at step 238, the secondary syringe and the primary syringe connected to the stopcock is leaved. Further, at step 240, the matrix blood is forced between the secondary syringe and the primary syringe to mix with air. Further, at step 242, the fibrin chains are broken into shorter lengths to form the Re-generative fibrin matrix. Further, at step 244, the Re-generative fibrin matrix through the secondary syringe is delivered.

Then the second syringe or the secondary syringe attached to the three-way stopcock, used to draw all of the air out of the cylinder with beads, pulling down blood through the cylinder 102 till it reaches second syringe. At which point the three-way stopcock is then turned to allowing third syringe or the tertiary syringe to draw blood from cylinder and first syringe or the primary syringe. When all of the blood is pulled out of first syringe or the primary syringe into the cylinder and into third syringe or the tertiary syringe the reverse process is reversed sending blood from third syringe or the tertiary syringe back into the cylinder then into first syringe or the primary syringe.

The reverse flow process is repeated between first syringe or the primary syringe and third syringe or the tertiary syringe for 10 number passes through the cylinder 102 housing the sintered beads 112. This process sheers or breaks the platelet membranes allow the release of fibrinogen to begin its formation to Fibrin.

Once the blood has passed 10 times between first or the primary syringe and third syringe or the tertiary syringe, then the stopcock 104 turned to flow the matrix blood into second syringe with air which was drawn from housed within from the cylinder at the beginning of this process.

After the matrix blood is passed into the second syringe the secondary syringe completely, the entire syringe is allowed to rest on a flat surface for 10 minutes. This resting time allows the transformation of the fibrinogen to transform to Fibrin, creating a Fibrin matrix from the blood. The Fibrin matrix is similar to a blood clot which is part of the healing component of blood in nature. After 10 minutes has passed and the matrix is solidified, then the system is raised to allow air and matrix in Second syringe to separate air on the top and matrix at the bottom.

The stopcock is opened to cylinder and air is forced back into the cylinder till all the air is removed from Second syringe. Then stopcock is opened allowing matrix to pass from Second syringe into Third syringe. At this point the cylinder and First syringe are disconnected at luer lock connect of leaving the second the secondary syringe and third syringes or the tertiary syringe connected to the stopcock. Then the matrix is forced 9 between Second syringe and Third syringe to break up the chains formed while matrix was mixed and rested with air. This action breaks the Fibrin chains into shorter lengths, so as to be able to inject the liquefied matrix through a 22-gauge needle.

After the matrix is liquefied and transferred completely into one of the two syringes it is ready for injection through a 22-gauge needle. If another material is desired to be added to this matrix, it would be added at this stage of processing by removing the empty syringe from the three-way stopcock and connecting another syringe housing the secondary material through the three-way stopcock into the syringe with the matrix a gentile shaking or swirling of syringe to ensure a homogeneous mixture. This process would be completed before injection is initiated.

While illustrative implementations of the application have been described in detail herein, it is to be understood that the inventive concepts may be otherwise variously embodied and employed, and that the appended claims are intended to be construed to include such variations, except as limited by the prior art.

Reference throughout this specification to “one implementation” or “an implementation” means that a particular feature, structure, or characteristic described in connection with the implementation is included in at least one implementation of the present invention. Thus, the appearances of the phrases “in one implementation” or “in some implementations” in various places throughout this specification are not necessarily all referring to the same implementation. Furthermore, the particular features, structures, or characteristics may be combined in any suitable manner in one or more implementations.

Systems and methods describing the present invention have been described. It will be understood that the descriptions of some embodiments of the present invention do not limit the various alternative, modified, and equivalent embodiments which may be include within the spirit and scope of the present invention as defined by the appended claims. Furthermore, in the detailed description above, numerous specific details are set forth to provide an understanding of various embodiments of the present invention. However, some embodiments of the present invention may be practiced without these specific details. In other instances, well known methods, procedures, and components have not been described in detail so as not to unnecessarily obscure aspects of the present embodiments.

1. A system for preparing and delivering Re-generative Fibrin Matrix, wherein the system comprising:
a cylinder with a first end and a second end, wherein the cylinder is filled with glass beads, further wherein the cylinder includes a male luer lock on each of the first end and the second end,
a three-way stopcock with a first way, a second way and a third way, wherein the first way of the three-way stopcock is inserted within the male luer lock on the first end of the cylinder; and
a plurality of syringes, wherein the plurality of syringes includes:
a first syringe, wherein the first syringe is connected to the male luer lock on the second end of the cylinder;
a second syringe, wherein the second syringe is connected to the second way of the three-way stopcock; and
a third syringe, wherein the third syringe is connected to the third way of the three-way stopcock.

2. The system in accordance with claim 1, wherein the plurality of glass beads are sintered beads.

3. The system in accordance with claim 1, wherein the plurality of glass beads are charged.

4. The system in accordance with claim 3, wherein the charge is negative or positive.

5. The system in accordance with claim 1, wherein the plurality of syringes are used for transferring the blood over and within the plurality of glass beads.

6. The system in accordance with claim 1, wherein each of the plurality of syringes includes a female luer lock.

7. The system in accordance with claim 6, wherein the female luer lock of the first syringe is connected to the male luer lock on the first end of the cylinder.

8. The system in accordance with claim 1, wherein the three-way stopcock is connected at the second end of cylinder between the male luer lock on the first end of the cylinder and the female luer lock the second syringe.

9. A method for preparation of Re-Generative Fibrin Matrix, wherein the method comprising:
arranging a cylinder, a stopcock and a number of syringes in a framework;
turning the stopcock towards a primary syringe;
drawing air from the cylinder through a secondary syringe;
pulling down blood through the cylinder till it reaches the secondary syringe;
turning the stopcock towards the secondary syringe;
allowing the primary syringe to draw the blood from the cylinder;
sending the blood from the primary syringe back to the cylinder;
transferring the blood from the cylinder into a tertiary syringe;
repeating the transfer of the blood between the tertiary syringe, the cylinder and the primary syringe for ten times for breaking platelet membrane and for releasing fibrinogen;
converting the fibrinogen to fibrin for forming a matrix blood;
turning the stopcock towards the cylinder;
flowing the matrix blood into the secondary syringe with air;
placing the framework on a flat surface for ten minutes once the matrix blood is passes completely in the secondary syringe;
uplifting the framework to allow mixing of air and the matrix blood in the secondary syringe;
separating air from the top end and the matrix blood at the bottom end of the secondary syringe;
opening the stopcock to the cylinder for forcing air back into the cylinder till the air in the secondary syringe is removed;
turning the stopcock towards the cylinder to allow the matrix blood;
passing the matrix blood from the secondary syringe to the primary syringe;
disconnecting the cylinder and the tertiary syringe at a luer lock connect;
leaving the secondary syringe and the primary syringe connected to the stopcock;
forcing the matrix blood between the secondary syringe and the primary syringe to mix with air;
breaking the fibrin chains into shorter lengths to form the Re-generative fibrin matrix; and
delivering the Re-generative fibrin matrix through the secondary syringe.

10. The method in accordance with claim 9, wherein the re-generative fibrin matrix is delivered through a 22-gauge needle.

11. The method in accordance with claim 9, wherein the re-generative fibrin matrix is a liquefied matrix.

12. The method in accordance with claim 11, wherein the liquefied matrix is added with a secondary material for diversified results.

13. The method in accordance with claim 12, wherein the method of adding the secondary material includes:
removing the tertiary syringe from the three-way stopcock;
connecting a syringe with the secondary material through the three-way stopcock;
pushing the secondary material from the syringe into the cylinder; and
swirling the syringe to form a homogeneous mixture.
